(19) United States
(12) Patent Application Publication
(10) Pub. No.: US 2019/0060529 A9
(43) Pub. Date: Feb. 28, 2019

(54) OXYGEN-CHARGED IMPLANTABLE MEDICAL DEVICES FOR AND METHODS OF LOCAL DELIVERY OF OXYGEN VIA OUTGASSING

(71) Applicant: THE JOHNS HOPKINS UNIVERSITY, BALTIMORE, MD (US)

(72) Inventors: COLIN ANDREW COOK, PASADENA, CA (US); WARREN L. GRAYSON, BALTIMORE, MD (US)

(73) Assignee: THE JOHNS HOPKINS UNIVERSITY, BALTIMORE, MD (US)

(21) Appl. No.: 15/011,990

(22) Filed: Feb. 1, 2016

Related U.S. Application Data
(63) Continuation-in-part of application No. 14/893,115, filed on Nov. 23, 2015.
(60) Provisional application No. 61/828,126, filed on May 28, 2013.

(51) Int. Cl. A61L 31/12; A61M 31/00; A61L 31/16

(52) U.S. Cl. CPC A61L 31/128; A61M 31/002; A61L 31/16; A61M 2202/0208; A61M 2202/0275; A61L 2430/34; A61M 2202/0216; A61M 2202/0291; A61L 2300/10; A61L 2300/114; A61L 2430/02; A61M 2202/0233

(57) ABSTRACT

Oxygen-charged (or oxygen-rich) implantable medical devices for and methods of local delivery of oxygen via outgassing is disclosed. The presently disclosed oxygen-charged implantable medical devices are, for example, polymeric implants that are functionalized to locally deliver oxygen from the implant surface for prolonged periods of time, thus increasing rates of healing and reducing rates of infection as compared with conventional medical implant devices.

### CROSS-REFERENCE TO RELATED APPLICATIONS

This application claims the benefit of U.S. Provisional Application No. 62/110,072, filed Jan. 30, 2015, which is incorporated herein by reference in its entirety.

### TECHNICAL FIELD

The presently disclosed subject matter relates generally to implantable medical devices and more particularly to oxygen-charged implantable medical devices and methods of local delivery of oxygen (or other gases) to a therapeutic site in a subject.

### BACKGROUND

Implantable medical devices (also called implants) are any devices that can be implanted in the body. Medical devices can be implanted into the body by several means, such as surgically, by injection using a syringe, by insertion using an endoscope, by ingestion, and the like. Examples of implantable medical devices include, but are not limited to, drug delivery implant devices, stents, catheters, replacement joints, orthopedic implants, craniofacial implants, prosthetics, valves, ocular implants, intraocular lenses, tissue engineering scaffolds, tissue transplant devices (e.g., islet encapsulation), tissue anchors, surgical fasteners (e.g., sutures, screws, pins, tacks, bolts, nails, suture anchors, staples, clips, and the like), and other related devices, such as rods, plates, meshes, foils, tethers, patches, slings, fabrics, conduits, tubes, and wires. Such devices are commonly used in bone-to-bone, soft tissue-to-bone, and/or soft tissue-to-soft tissue fixation. These devices can be formed of metal, metal alloys, polymers, non-bioresorbable high strength plastic materials, and/or bioresorbable materials, such as bioresorbable polymers.

A drawback of using implantable medical devices is that the implantation of the devices into the body can damage local vasculature/tissue and further risks introducing pathogens to the wound site. The hypoxic environment, i.e., an environment deprived of an adequate oxygen supply, which results can compromise healing and facilitate the establishment of bacterial colonies and biofilms because of the reduced potency of antibiotics and less leukocyte oxidative killing. Oxygen delivery remains one of the greatest challenges in tissue engineering. Consequently, many technologies and approaches have been developed, but each has met with limited success.

### SUMMARY

In some aspects, the presently disclosed subject matter provides an implantable medical device comprising one or more gases dissolved in one or more materials. In particular aspects, the device comprises one or more gases dissolved in one or more polymeric materials. In certain aspects, the polymeric material can be functionalized. In more certain aspects, the material can be hyperbarically-charged with one or more gases. In yet more certain aspects, the one or more gases comprise oxygen. In other aspects, the one or more materials comprise a plurality of pores adapted to store one or more gases.

In other aspects, the presently disclosed subject matter provides a method for delivering one or more gases to a therapeutic target, the method comprising: providing an implantable medical device comprising one or more gases dissolved in one or more materials; and implanting the device in the proximity of a therapeutic target in a subject thereof. In such methods, the one or more gases can comprise one or more additional components, wherein the one or more additional components are selected from the group consisting of a metabolic component, a signaling molecule, and an antimicrobial agent.

In particular aspects, the device is selected from the group consisting of a drug delivery implant device, a stent, a catheter, a replacement joint, an orthopedic implant, a craniofacial implant, a prosthesis, a valve, an ocular implant, an intraocular lens, a tissue engineering scaffold, tissue transplant devices (e.g., islet encapsulation), a tissue anchor, a surgical fastener, including a suture, a screw, a pin, a tack, a bolt, a nail, a suture anchor, a staple, a clip, and the like, and other related devices, such as a rod, a plate, a mesh, a foil, a tether, a patch, a sling, a fabric, a conduit, a tube, and a wire.

Certain aspects of the presently disclosed subject matter having been stated hereinabove, which are addressed in whole or in part by the presently disclosed subject matter, other aspects will become evident as the description proceeds when taken in connection with the accompanying Example and Drawings as best described herein below.

### BRIEF DESCRIPTION OF THE DRAWINGS

Having thus described the presently disclosed subject matter in general terms, reference will now be made to the accompanying Drawings, which are not necessarily drawn to scale, and wherein:

FIG. 1A and FIG. 1B illustrate a perspective view and a cross-sectional view, respectively, of an example of the presently disclosed oxygen-charged implantable medical device;

FIG. 2 illustrates a flow diagram of an example of a method of making the presently disclosed oxygen-charged implantable medical devices;

FIG. 3 illustrates a flow diagram of an example of a method of delivering oxygen to tissue using the presently disclosed oxygen-charged implantable medical devices; and

FIG. 4 is a photograph of a plurality of microballoons in a polymer, e.g., polycaprolactone (PCL).

### DETAILED DESCRIPTION

The presently disclosed subject matter now will be described more fully hereinafter with reference to the accompanying Drawings, in which some, but not all embodiments of the presently disclosed subject matter are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Drawings. Therefore, it is to be understood that the presently disclosed subject matter is not to be limited to the specific embodiments disclosed and that modifications and other embodiments are intended to be included within the scope of the appended claims.

In some embodiments, the presently disclosed subject matter provides oxygen-charged (or oxygen-rich) implantable medical devices for delivery of oxygen via outgassing to a targeted therapeutic site in a subject in need of treatment thereof. As used herein, the term “charged” includes impregnating, enriching, and/or permeating one or more materials, such as a polymer, with one or more gases, such that the one or more gases are dissolved in the one or more materials.

The presently disclosed oxygen-charged implantable medical devices are, for example, polymeric implants that can be functionalized to locally deliver oxygen from the implant surface for prolonged periods of time, thus increasing rates of healing and reducing rates of infection as compared with conventional medical implant devices.

Further, the presently disclosed oxygen-charged implantable medical device is an example of using controlled outgassing of hyperbarically-loaded polymeric materials for the delivery of oxygen and/or other therapeutic gases in biomedical applications. For example, oxygen is dissolved in the implantable medical devices and then the device is implanted into the body of a subject, wherein the oxygen is released via outgassing in a controlled and sustained manner.

Examples of the oxygen-charged implantable medical devices include, but are not limited to, oxygen-charged drug delivery implant devices, stents, catheters, replacement joints, orthopedic implants, craniofacial implants, prosthetics, valves, ocular implants, intraocular lenses, tissue engineering scaffolds, tissue transplant devices (e.g., islet encapsulation), tissue anchors, surgical fasteners (e.g., sutures, screws, pins, tacks, bolts, nails, suture anchors, staples, clips, and the like), and other related devices, such as oxygen-charged rods, plates, meshes, foils, tethers, patches, slings, fabrics, conduits, tubes, and wires.

The presently disclosed oxygen-charged implantable medical devices can be used, for example, in bone-to-bone fixation, soft tissue-to-bone fixation, and/or soft tissue-to-soft tissue fixation.

The implantable medical devices disclosed herein are not limited to being charged with oxygen only. The implantable medical devices can be charged with one or more types of gases including oxygen. In some embodiments, the one or more gases are selected from the group consisting of oxygen, nitric oxide, carbon monoxide, carbon dioxide, hydrogen, hydrogen sulfide, ozone, xenon, ethylene, a sulfite, and combinations thereof. In other embodiments, the one or more gases comprise a metabolic component. In yet other embodiments, the one or more gases comprise oxygen. In yet other embodiments, the one or more gases comprise a signaling molecule. In other embodiments, the signaling molecule comprises a vasodilator. In yet other embodiments, the one or more gases comprise nitric oxide. In still other embodiments, the one or more gases comprise an antimicrobial agent. In still other embodiments, the one or more gases comprise ozone.

Further, the presently disclosed subject matter provides a method of making the oxygen-charged implantable medical devices, wherein oxygen and/or one or more other gases is dissolved within the bulk polymer. Additionally, the presently disclosed subject matter provides a method of local delivery of oxygen to surrounding tissue via outgassing. In some embodiments, the oxygen is delivered to hypoxic tissue.

An aspect of the presently disclosed oxygen-charged implantable medical devices and methods is that it provides the nondestructive/noninvasive functionalization of new or existing implants. Namely, oxygen delivery can be added without changing the manufacturing, composition, geometry, mechanical properties of the implants by merely functionalizing existing polymeric implants.

Another aspect of the presently disclosed oxygen-charged implantable medical devices and methods is that it provides a substantially pure oxygen delivery method that is substantially free from carriers, byproducts, and/or intermediates.

Yet another aspect of the presently disclosed oxygen-charged implantable medical devices and methods is that it provides rapid functionalization and facile long-term storage of functionalized implants. In some embodiments, the presently disclosed oxygen-charged implantable devices can be stored, for example, at reduced temperatures, e.g., between about −20° C. to about −80° C., and/or maintained under pressure until needed.

Yet another aspect of the presently disclosed oxygen-charged implantable medical devices and methods is that it provides local potentiation of antibiotics and anti-anaerobic properties.

Yet another aspect of the presently disclosed oxygen-charged implantable medical devices and methods is that, in addition to oxygen, other gases can be delivered in a similar manner for other applications.

Still another aspect of the presently disclosed oxygen-charged implantable medical devices and methods is that it uses outgassing for therapeutic purposes.

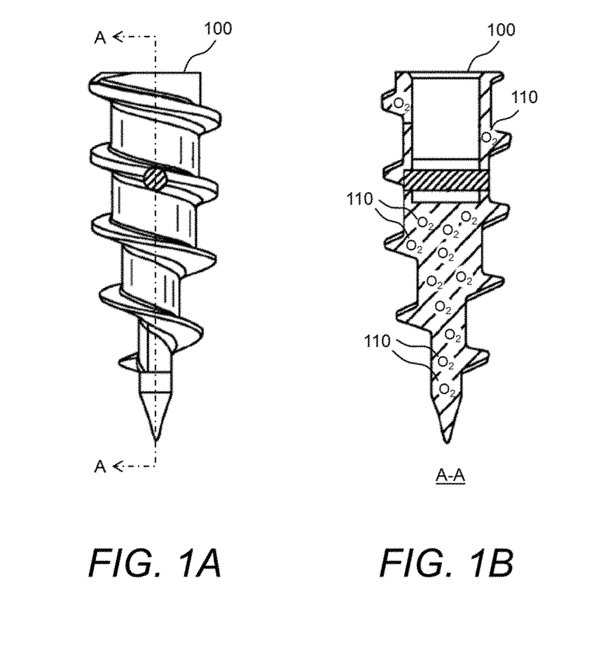

Referring now to FIG. 1A is a perspective view of an example of the presently disclosed oxygen-charged implantable medical device 100, while FIG. 1B is a cross-sectional view of the oxygen-charged implantable medical device 100 taken along line A-A of FIG. 1A. In this example, the oxygen-charged implantable medical device 100 is an oxygen-charged suture anchor. A suture anchor, however, is exemplary only. The oxygen-charged implantable medical device 100 can be any type of implantable device, such as, but not limited to, oxygen-charged drug delivery implant devices, stents, catheters, replacement joints, craniofacial implants, prosthetics, valves, ocular implants, intraocular lenses, tissue engineering scaffolds, tissue transplant devices (e.g., islet encapsulation), tissue anchors, surgical fasteners (e.g., sutures, screws, pins, tacks, bolts, nails, suture anchors, staples, clips, and the like), and other related devices, such as oxygen-charged rods, plates, meshes, foils, tethers, patches, slings, fabrics, conduits, tubes, and wires.

The oxygen-charged implantable medical device 100 can be, for example, an oxygen-charged polymeric implant device, meaning an implant device that is formed of a polymer material that has a high concentration of oxygen therein. For example, FIG. 1B shows a quantity of oxygen (O₂) molecules 110 in the polymer material that forms the oxygen-charged implantable medical device 100.

In some embodiments, the presently disclosed oxygen-charged implantable medical devices are polymeric implants or implants with polymeric components. The polymer can be any polymer having a solubility for one or more gases, including oxygen. Examples of polymers suitable for use with the presently disclosed matter include, but are not limited to, polyvinyl alcohol (PVA), polylactic acid (PLA), ethylene vinyl alcohol (EVOH), poly(lactide-co-glycolide) (PLGA), polyglycolide (PGA), nylon, polyketone, polyether ether ketone (PEEK), polyethylene terephthalate (PET), polyvinylidine chloride (PVDC), polyacrylonitrile (PAN), polyamides (PAs), polyvinyl chloride (PVC), polyvinylidene fluoride (PVDF), polyethylenimine (PEI), polycarbonate (PC), ethylene chlorotrifluoroethylene (ECTFE), polyethylene naphthalene (PEN), polytrimethylene terephthalate (PTT), liquid crystal polymers (e.g., Kevlar), nanocellulose, poly(methylmethacrylate (PMMA), poly(p-xylylene) polymers (e.g., PARYLENE® C, D, HT, AM, N), and polybutylene terephthalate (PBT).

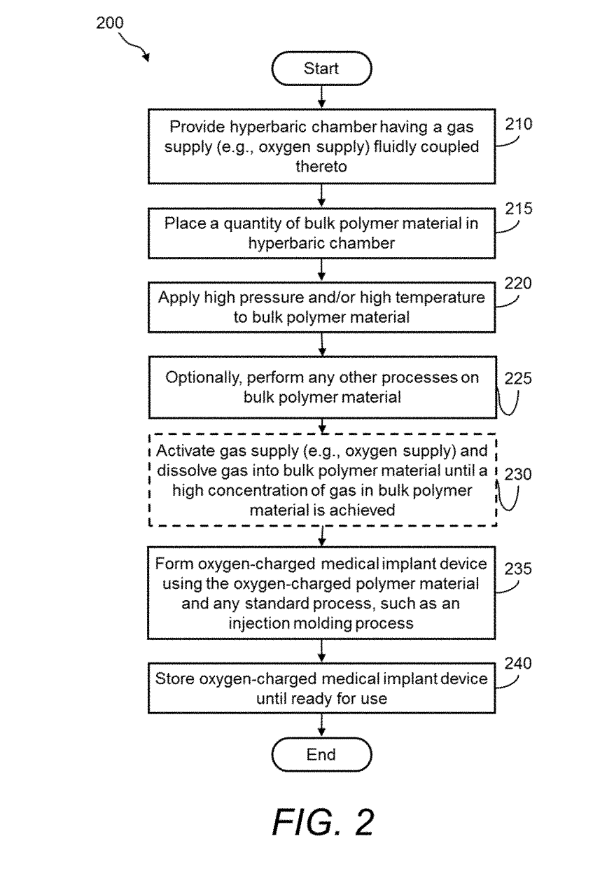

Further, the presently disclosed oxygen-charged implantable medical device, such as the oxygen-charged implantable medical device 100 shown in FIG. 1A and FIG. 1B provides a means of functionalizing new or existing implants with oxygen delivery capability of meaningful capacity and duration. The oxygen-charged implantable medical devices are hyperbarically loaded with oxygen using a hyperbaric chamber, as described herein below with reference to FIG. 2, and upon removal from the chamber, outgases the oxygen from the implant surface, thereby providing prolonged local oxygen delivery. This characteristic is achieved by taking advantage of two innate properties of polymers: (1) non-trivial solubility of oxygen within polymers and (2) low diffusion coefficient of oxygen within polymers.

The property of oxygen solubility relates to the oxygen capacity or amount of oxygen that the polymer can contain. The diffusivity, solubility, and permeability of gases in polymers is a function of several factors, including, but not limited to, the molecular size and physical state of the gas; the morphology of the polymer; the compatibility or solubility limit of the gas within the polymer matrix; and the volatility of the gas. It is established that the concentration of oxygen within a polymer is proportional to the solubility of oxygen and the pressure of oxygen around the polymer.

Many polymers exhibit a solubility for oxygen on the order of about 0.1% to about 10% V/V/atm. Namely, polymers contain about 0.1-10% the amount of oxygen as would be contained in the same volume of gaseous oxygen at a given pressure, including 0.1%, 0.5%, 1.0%, 1.5%, 2.0%, 2.5%, 3.0%, 3.5%, 4.0%, 4.5%, 5.0%, 5.5%, 6.0%, 6.5%, 7.0%, 7.5%, 8.0%, 8.5%, 9.0%, 9.5%, and 10%. The presently disclosed subject matter leverages this property by hyperbarically loading polymers with oxygen to achieve multiples more oxygen content than is present at ambient pressure. The maximum loading pressure is limited by the mechanical properties of the polymer, which for most polymers falls within the range of from about 100 atm to about 1000 atm. Thus significant oxygen capacity within the polymer can be achieved.

The oxygen diffusion coefficient in polymers relates to the period/duration of oxygen delivery. Oxygen contained within a polymer will tend to diffuse, as described by Fick's Law, from regions of high concentration to regions of low concentration at a rate prescribed by the coefficient of diffusion. Fick's first law of diffusion states that gases will tend to diffuse from regions of higher partial pressures to regions of lower partial pressures. Hence, when initially placed in a hyperbaric chamber, gases will tend to permeate into the bulk polymer material. Upon removal from the hyperbaric chamber, gases will tend to exit the polymer material, i.e., referred to herein as “outgassing.”

In polymers, where the coefficient of diffusion is low, oxygen will diffuse slowly and thus provide a means of prolonging the delivery of oxygen. For a polymeric implant that is initially loaded with oxygen (e.g., the oxygen-charged implantable medical device 100) and then placed in the body, oxygen will gradually diffuse out of the surfaces of the implant thereby enriching the local environment with oxygen. The period of time over which the oxygen delivery will occur is approximately proportional to the square of the thickness of the implant and inversely proportional to the diffusion coefficient of oxygen within the polymer. For many existing medical polymers and implants, this yields a delivery period of from about 1 week to about 4 weeks. Depending on the polymers selected and the geometry of the implant, the delivery period can range from minutes months. Thus, significant periods of oxygen delivery can be achieved.

Given that different polymers exhibit a range of coefficients of diffusion of oxygen (or other gases) and a range of solubilities for oxygen (or other gases), it is possible to create laminated or otherwise multi-material implants that exhibits hybrid behaviors. For example, a coating of a gas barrier layer can be made around an implant to increase the overall period of gas, e.g., oxygen, delivery. The gas barrier layer can include, but not be limited to, a high oxygen barrier polymer, SiOₓ, silica, Al₂O₃, one or more metals, a nanocomposite, and the like.

Additionally, a polymer having a high solubility for oxygen (or other gases) can be added within an implant to provide overall increased oxygen capacity of the implant. As an example, a medical implant with a short period of outgassing can be modified by adding a coating of poly(p-xylylene) polymers, e.g., PARYLENE®, through chemical vapor deposition. The poly(p-xylylene) coating increases the period of outgassing of the implant in a manner proportional to the thickness of the coating layer. PARYLENE® C in particular provides excellent gas barrier properties. Poly(p-xylylenes) can provide uniform, pinhole-free, conformal coatings and are biocompatible making them particularly useful polymers for outgassing applications. Such coatings, in some embodiments, can have a thickness ranging from about 1 micron to about 10 microns, and, in other embodiments, a thickness ranging from about 10 microns to about 1000 microns.

Oxygen release from the presently disclosed oxygen-charged implantable medical devices enhances wound healing and prevents infection. Elevated oxygen levels have been shown to increase vasculogenesis and collagen deposition, leading to improved healing outcomes. Elevated oxygen prevents the growth of anaerobic bacteria; oxygen potentiates antibiotics, enhancing the killing of aerobic bacteria. Elevated oxygen levels also produce a more robust oxidative killing response by leukocytes. Elevated oxygen enhances the immune system response. In a healthcare system facing increasing incidence of antibiotic-resistant infections, the antimicrobial benefits of local oxygen delivery are significant. The combined benefits of local oxygen delivery from the presently disclosed oxygen-charged implantable medical devices can reduce the risk of implant failure.

As an additive technology, the presently disclosed oxygen-charged implantable medical devices and methods allow existing products to achieve greater therapeutic value without the need to re-design and re-manufacture.

Referring now to FIG. 2 is a flow diagram of an example of a method 200 of making the presently disclosed oxygen-charged implantable medical devices 100. The method 200 may include, but is not limited to, the following steps.

At a step 210, a hyperbaric chamber having a gas supply (e.g., oxygen supply) fluidly coupled thereto is provided. In one example, a hyperbaric chamber is provided that is capable of producing up to about 100 atm of pressure and that is capable of heating up to about 100° C.

At a step 215, a quantity of bulk polymer material is placed in the hyperbaric chamber. In one example, the bulk polymer material is provided in a solid state.

At a step 220, high pressure and/or high temperature is applied to the bulk polymer material. In one example, the applied pressure is from about 100 atm to about 1000 atm. In another example, the applied pressure is about 10 atm. In one example, the temperature can be from about 50° C. to about 200° C. In another example, the temperature is about 100° C.

At an optional step 225, any other processes are performed on the bulk polymer material. For example, to further enhance the oxygen capacity of the polymer, gas-containing (e.g., oxygen-containing) microtanks, pores, foaming, and the like can be added to the polymer to achieve meaningful concentrations of oxygen. Examples of gas-containing (e.g., oxygen-containing) microtanks are described with reference to International Application No. PCT/US14/39806, entitled “Controlled Outgassing of Hyperbarically Loaded Materials for the Delivery of Oxygen and Other Therapeutic Gases in Biomedical Applications,” filed on May 28, 2014, and published as International Application Publication No. WO/2014/193963, published Dec. 4, 2014; the entire disclosure of which is incorporated herein by reference.

At a step 230, the gas supply (e.g., oxygen supply) is activated and the gas is dissolved into bulk polymer material until a high concentration of gas in the bulk polymer material is achieved. Typically the loading is an approximately exponential process so within three time constants of loading the implant contains a concentration of 95% of the equilibrium concentration.

At a step 235, the oxygen-charged medical implant device is formed using the oxygen-charged (or oxygen-rich) polymer material and any standard processes, such as an injection molding process, although typically the loading is achieved by placing the medical implant device in a hyperbaric chamber and charging with oxygen, or one or more gases, before implantation.

At a step 240, the oxygen-charged medical implant device is stored until ready for use. The shelf life and storage requirements are closely related. At reduced temperatures, the rate of outgassing from the device decreases exponentially. The recommended shelf-life is therefore related to the allowable deviation in concentration of gas from the freshly charged state, for example 10%-30%. Once an allowable deviation is established, the recommended shelf life is given by the time required for the device to outgas this amount of oxygen at the given storage conditions. For common polymers, the use of storage conditions at −20° C. and −80° C. can increase the time constant of outgassing from about 35 to 15,000 times, respectively. Thus meaningful shelf lives can be achieved on the order of weeks to years. Additionally, implants that are stored under hyperbaric oxygen conditions will maintain oxygen content indefinitely.

In further embodiments, to enhance the solubility of one or more gases within an implantable medical device, the bulk polymer comprising the medical device can be mixed with one or more void-forming particles to create porosity in the bulk polymer. For example, a polymer, such as a biodegradable thermoplastic, such as polylactic acid (PLA) can be mixed, e.g., melt-mixed, with a plurality of glass microballoons and injected into a mold for forming the medical device, e.g., an implantable screw. The polymer can then be allowed to cool and to solidify. In other embodiments, the bulk polymer can be melted with a solvent. In other embodiments, the polymer can comprise an epoxy, in which void-forming particles can be mixed into before curing.

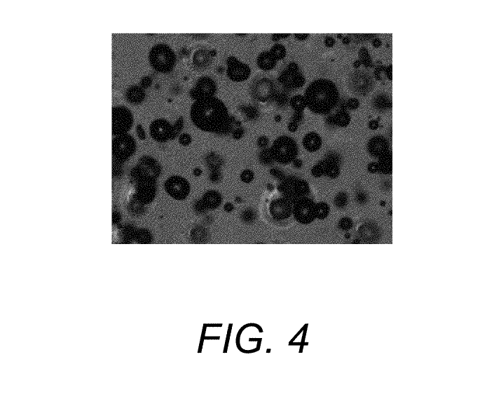

In such embodiments, the glass microballoon can have a particular hydrostatic pressure at which they will crack or rupture. By hyperbarically loading the polymer at pressures in excess of this hydrostatic pressure limit, the embedded glass microballoons crack or rupture and become permeable voids where oxygen can be stored. The use of glass microballoons allows for the generation of voids into high-melting temperature polymers and materials. In this manner, additional oxygen can be stored within the medical implant compared to the amount dissolved in the bulk polymer alone. An example of a polymer embedded with a plurality of microballoons is provided in FIG. 4.

As used herein, the term “microballoon” is a subset of what is referred to as a “microtank,” see International Application Publication No. WO/2014/193963, referenced herinabove. A microtank can refer to as any void, wherein a microballoon comprises a shell that is self supporting.

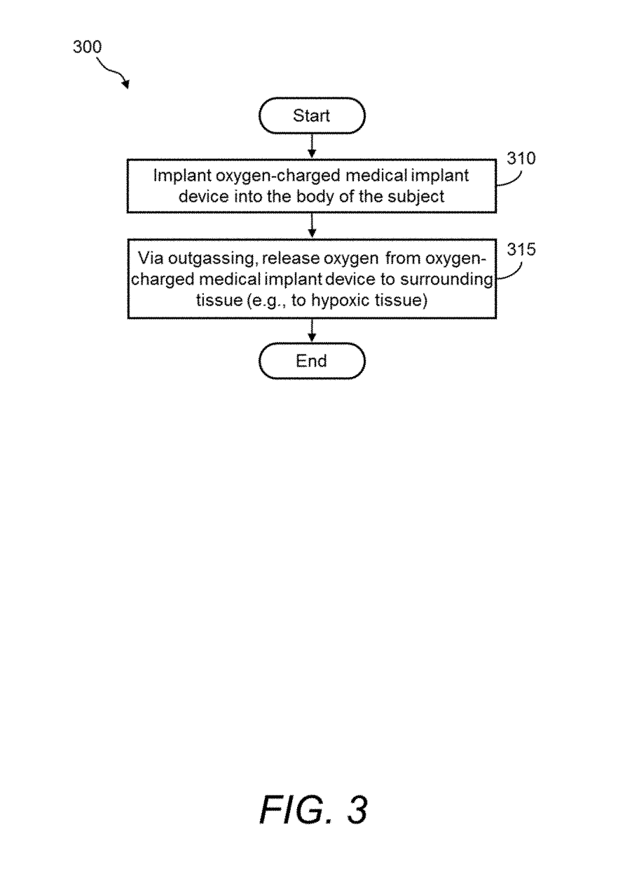

Referring now to FIG. 3 is a flow diagram of an example of a method 300 of delivering oxygen to tissue using the presently disclosed oxygen-charged implantable medical devices 100. Namely, the method 300 uses the presently disclosed oxygen-charged implantable medical devices 100 to locally deliver oxygen from the implant surface to the surrounding tissue. The method 300 may include, but is not limited to, the following steps.

At a step 310, the oxygen-charged medical implant device (e.g., the oxygen-charged implantable medical device 100) is implanted into the body of the subject. For example, the oxygen-charged medical implant device can be implanted into the body by any means, such as surgically, by injection using a syringe, by insertion using an endoscope, by ingestion, and so on.

At a step 315, via outgassing, oxygen is released from the surface of the oxygen-charged medical implant device (e.g., the oxygen-charged implantable medical devices 100) to surrounding tissue (e.g., to hypoxic tissue). In so doing, oxygen is delivered locally from the surface of the oxygen-charged medical implant device to the surrounding tissue, wherein the oxygen may be delivered for prolonged periods of time (e.g., from about 1 hour to about 4 weeks), thus increasing rates of healing and reducing rates of infection as compared with conventional medical implant devices.

Referring again to FIG. 1A, FIG. 1B, FIG. 2, and FIG. 3, the presently disclosed subject matter provides oxygen-charged medical implant devices (e.g., the oxygen-charged implantable medical device 100) for delivering one or more gases in a controlled and sustained manner to surrounding tissue. In some embodiments, the oxygen is delivered to hypoxic tissue. Further, the presently disclosed oxygen-charged medical implant devices can be applied to tissue engineering and the mitigation of ischemia and hypoxia, among other applications.

In the oxygen-charged medical implant devices and methods, the use of controlled outgassing of hyperbarically loaded materials for the delivery of oxygen and other therapeutic gases in biomedical applications provides numerous advantages over the prior art including, but not limited to, controlling the release profile of the gases, the dose of the gases, the spatial distribution of the gases, and the components of the gases.

These aspects of the oxygen-charged medical implant devices and methods allow for gases to be used in a completely novel and useful manner. Currently, using methods known in the art, gases are delivered systemically through ventilation, which limits their therapeutic applications. In contrast, the presently disclosed oxygen-charged medical implant devices and methods provide for a controllable localization, release profile, and dosage. Accordingly, the therapeutic utility and potential of gases delivered in this way increases greatly, analogous to how the controlled release of proteins and drugs has enhanced their utility. The subject treated by the presently disclosed devices and methods in their many embodiments is desirably a human subject, although it is to be understood that the methods described herein are effective with respect to all vertebrate species, which are intended to be included in the term “subject.”

A “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLE

The following Example has been included to provide guidance to one of ordinary skill in the art for practicing representative embodiments of the presently disclosed subject matter. In light of the present disclosure and the general level of skill in the art, those of skill can appreciate that the following Example is intended to be exemplary only and that numerous changes, modifications, and alterations can be employed without departing from the scope of the presently disclosed subject matter. The following Example is offered by way of illustration and not by way of limitation.

### Example 1

### Porous Polymers for Storing Gases in Implantable Medical Devices

To enhance the oxygen solubility within medical implants, the bulk polymers can be mixed with void forming particles to create porosity. This approach was demonstrated in the formation of a biodegradable screw. Polylactic acid (PLA) was melt-mixed with glass microballoons and injected into a screw mold where it was cooled and allowed to solidify. The glass microballoons have a designated hydrostatic pressure at which they crack. By hyperbarically loading the screw at pressures in excess of this hydrostatic pressure limit, the glass microballoons embedded in the bulk polymer of the screw crack and become permeable voids where oxygen can be stored. The use of glass microballoons allows for the generation of voids into high-melting temperature polymers and materials. In this manner, additional oxygen can be stored within the medical implant compared to just the amount dissolved in the bulk polymer.

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.

International Application No. PCT/US14/39806, entitled “Controlled Outgassing of Hyperbarically Loaded Materials for the Delivery of Oxygen and Other Therapeutic Gases in Biomedical Applications,” filed on May 28, 2014.

Although the foregoing subject matter has been described in some detail by way of illustration and example for purposes of clarity of understanding, it will be understood by those skilled in the art that certain changes and modifications can be practiced within the scope of the appended claims.

That which is claimed:

1. An implantable medical device comprising one or more gases dissolved in one or more materials.

2. The implantable medical device of claim 1, comprising one or more gases dissolved in one or more polymeric materials.

3. The implantable medical device of claim 2, wherein the one or more polymeric materials are functionalized.

4. The implantable medical device of claim 1, wherein the material is hyperbarically-charged with one or more gases.

5. The implantable medical device of claim 2, wherein the one or more polymeric materials is selected from the group consisting of polyvinyl alcohol (PVA), polylactic acid (PLA), ethylene vinyl alcohol (EVOH), poly(lactide-co-glycolide) (PLGA), polyglycolide (PGA), nylon, polyketone, polyether ether ketone (PEEK), polyethylene terephthalate (PET), polyvinylidine chloride (PVDC), polyacrylonitrile (PAN), polyamides (PAs), polyvinyl chloride (PVC), polyvinylidene fluoride (PVDF), polyethylenimine (PEI), polycarbonate (PC), ethylene chlorotrifluoroethylene (ECTFE), polyethylene naphthalene (PEN), polytrimethylene terephthalate (PTT), liquid crystal polymers (e.g., Kevlar), nanocellulose, poly(methylmethacrylate (PMMA), polybutylene terephthalate (PBT), poly(p-xylylene), and derivatives thereof.

6. The implantable medical device of claim 1, wherein the one or more gases are selected from the group consisting of oxygen, nitric oxide, carbon monoxide, carbon dioxide, hydrogen, hydrogen sulfide, ozone, xenon, ethylene, a sulfite, and combinations thereof.

7. The implantable medical device of claim 6, wherein the one or more gases comprises oxygen.

8. The implantable medical device of claim 1, wherein the one or more gases comprises one or more additional components.

9. The implantable medical device of claim 8, wherein the one or more additional components is selected from the group consisting of a metabolic component, a signaling molecule, and an antimicrobial agent.

10. The implantable medical device of claim 9, wherein the signaling molecule comprises a vasodilator.

11. The implantable medical device of claim 1, wherein the device is selected from the group consisting of a drug delivery device, a stent, a catheter, a replacement joint, an orthopedic implant, a craniofacial implant, a prosthesis, a valve, an ocular implant, an intraocular lens, a tissue engineering scaffold, a tissue transplant device, a tissue anchor, a surgical fastener, a rod, a plate, a mesh, a foil, a tether, a patch, a sling, a fabric, a conduit, a tube, and a wire.

12. The implantable medical device of claim 11, wherein the surgical fastener is selected from the group consisting of a suture, screw, a pin, a tack, a bolt, a nail, a staple, a clip, and combinations thereof.

13. The implantable medical device of claim 1, wherein the device is adapted for bone-to-bone fixation, soft tissue-to-bone fixation, soft tissue-to-soft tissue fixation, and combinations thereof.

14. The implantable medical device of claim 1, wherein the one or more materials comprise a plurality of pores adapted to store one or more gases.

15. The implantable medical device of claim 1, wherein the implantable medical device is coated with a gas barrier layer.

16. The implantable medical device of claim 1, wherein the implantable medical device is coated with coating having a high solubility for the one or more gases.

17. A method for delivering one or more gases to a therapeutic target, the method comprising:
providing an implantable medical device comprising one or more gases dissolved in one or more materials; and
implanting the device in the proximity of a therapeutic target in a subject thereof.

18. The method of claim 17, wherein the implantable medical device comprises one or more gases dissolved in one or more polymeric materials.

19. The method of claim 18, wherein the one or more polymeric materials are functionalized.

20. The method of claim 17, wherein the material is hyperbarically-charged with one or more gases.

21. The method of claim 18, wherein the one or more polymeric materials is selected from the group consisting of polyvinyl alcohol (PVA), polylactic acid (PLA), ethylene vinyl alcohol (EVOH), poly(lactide-co-glycolide) (PLGA), polyglycolide (PGA), nylon, polyketone, polyether ether ketone (PEEK), polyethylene terephthalate (PET), polyvinylidine chloride (PVDC), polyacrylonitrile (PAN), polyamides (PAs), polyvinyl chloride (PVC), polyvinylidene fluoride (PVDF), polyethylenimine (PEI), polycarbonate (PC), ethylene chlorotrifluoroethylene (ECTFE), polyethylene naphthalene (PEN), polytrimethylene terephthalate (PTT), liquid crystal polymers (e.g., Kevlar), nanocellulose, poly(methylmethacrylate (PMMA), polybutylene terephthalate (PBT), poly(p-xylylene) and derivatives thereof.

22. The method of claim 17, wherein the one or more gases are selected from the group consisting of oxygen, nitric oxide, carbon monoxide, carbon dioxide, hydrogen, hydrogen sulfide, ozone, xenon, ethylene, a sulfite, and combinations thereof.

23. The method of claim 22, wherein the one or more gases comprises oxygen.

24. The method of claim 17, wherein the one or more gases comprises one or more additional components.

25. The method of claim 24, wherein the one or more additional components is selected from the group consisting of a metabolic component, a signaling molecule, and an antimicrobial agent.

26. The method of claim 25, wherein the signaling molecule comprises a vasodilator.

27. The method of claim 17, wherein the device is selected from the group consisting of a drug delivery device, a stent, a tissue anchor, a surgical fastener, an ocular implant, a tissue engineering scaffold, a tissue transplant device, a rod, a plate, and a wire.

28. The method of claim 27, wherein the surgical fastener is selected from the group consisting of a suture, screw, a pin, a tack, a bolt, a nail, a staple, a clip, and combinations thereof.

29. The method of claim 17, wherein the device is adapted for bone-to-bone fixation, soft tissue-to-bone fixation, soft tissue-to-soft tissue fixation, and combinations thereof.
